SEQUENCE LISTING (4 sequences)

SEQ ID NO: 1 - DNA, 1500 bp, Clostridium sp.
gtcgagcggttctgcattcaacattgagtattcagctgacgttggagattgagccggcgtcaagcagaaacaaccatatactatgatatgtttaaagtttctgcttcacgctgaataaagtgccaacacaaaaaggagctgagtattgaatgttgaatgcaggatagcggcggacgggtgagtaacgcgtgggtaatctgcctcacacagggggataacactgggaaactagtgctaataccgcataacataacgaggtggcatcactttgttatcaaaggagcaatccggtgtgagatgagcccgcgtccgattagctagttggtgaggtaacggctcaccaaggcgacgatcggtagccgaactgagaggttgatcggccacattgggactgagacacggcccagactcctacgggaggcagcagtggggaatattgcgcaatgggggaaaccctgacgcatcaacgccgcgtgaaggaagaaggtcttcggattgtaaacttctttggttggggacgataatgacggtacccaaggaacaagccacggctaactacgtgccagcagccgcggtaatacgtaggtggcgagcgttgtccggaattactgggtgtaaagggcgcgtaggcggggatgcaagtcagatgtgaaattccggggctcaaccccggcgctgcatctgaaactgtatttcttgagtgctggagaggaaagcggaattcctagtgtagcggtgaaatgcgtagagattaggaggaacaccagtggcgaaggcggctttctggacagtaactgacgctgaggcgcgaaagcgtggggagcaaacaggattagataccctggtagtccacgccgtaaacgatggatactaggtgtaggaggtatcgaccccttctgtgccggagttaacacaataagtatcccacctggggagtacggccgcaaggttgaaactcaaaggaattgacgggggcccgcacaagcagtggagtatgtggtttaattcgaagcaacgcgaagaaccttaccagggcttgacatccctctgacagctctagagatagggcttctcttcggagcagaggagacaggtggtgcatggttgtcgtcagctcgtgtcgtgagatgttgggttaagtcccgcaacgagcgcaacccttgttgttagttgccatcattaagttgggcactctaacgagactgccggtgataaatcggaggaaggtggggacgacgtcaaatcatcatgccccttatgtcctgggctacacacgtactacaatggctgctacaaagggaagcgagaccgcgaggtggagcaaatccccaaaagcagtcccagttcagattgcaggctgaaactcgcctgcatgaagtcggaattgctagtaatggcaggtcagcatactgccgtgaatacgttcccgggccttgtacacaccgcccgtcacaccatgagagtctgcaacacccgaagtcagtagtctaaccgagagga

SEQ ID NO: 2 - DNA, 1508 bp, Clostridium sp.
tgcaagtcgagcggttctgcattcaacattgagtattcagctgacgttggagattgagccggcgtcaagcagaaacaaccatatactatgatatgtttaaagtttctgcttcacgctgaataaagtgccaacacaaaaaggagctgagtattgaatgttgaatgcaggatagcggcggacgggtgagtaacgcgtgggtaatctgcctcacacagggggataacactgggaaactagtgctaataccgcataacataacgaggtggcatcactttgttatcaaaggagcaatccggtgtgagatgagcccgcgtccgattagctagttggtgaggtaacggctcaccaaggcgacgatcggtagccgaactgagaggttgatcggccacattgggactgagacacggcccagactcctacgggaggcagcagtggggaatattgcgcaatgggggaaaccctgacgcatcaacgccgcgtgaaggaagaaggtcttcggattgtaaacttctttggttggggacgataatgacggtacccaaggaacaagccacggctaactacgtgccagcagccgcggtaatacgtaggtggcgagcgttgtccggaattactgggtgtaaagggcgcgtaggcggggatgcaagtcagatgtgaaattccggggctcaaccccggcgctgcatctgaaactgtatttcttgagtgctggagaggaaagcggaattcctagtgtagcggtgaaatgcgtagagattaggaggaacaccagtggcgaaggcggctttctggacagtaactgacgctgaggcgcgaaagcgtggggagcaaacaggattagataccctggtagtccacgccgtaaacgatggatactaggtgtaggaggtatcgaccccttctgtgccggagttaacacaataagtatcccacctggggagtacggccgcaaggttgaaactcaaaggaattgacgggggcccgcacaagcagtggagtatgtggtttaattcgaagcaacgcgaagaaccttaccagggcttgacatccctctgacagctctagagatagggcttctcttcggagcagaggagacaggtggtgcatggttgtcgtcagctcgtgtcgtgagatgttgggttaagtcccgcaacgagcgcaacccttgttgttagttgccatcattaagttgggcactctaacgagactgccggtgataaatcggaggaaggtggggacgacgtcaaatcatcatgccccttatgtcctgggctacacacgtactacaatggctgctacaaagggaagcgagaccgcgaggtggagcaaatccccaaaagcagtcccagttcagattgcaggctgaaactcgcctgcatgaagtcggaattgctagtaatggcaggtcagcatactgccgtgaatacgttcccgggccttgtacacaccgcccgtcacaccatgagagtctgcaacacccgaagtcagtagtctaaccgagaggaggg

SEQ ID NO: 3 - DNA, 1062 bp, Clostridium sp.
ggtcatcagaccacaagtgaagccttcagttacatggtttggctgggtgcaacctatggcaaattgacgggagactggtcttattataaaaatgcatgggatttaaccgaaaaatacataattcccgatgcccaaaaagaccaaccgggtgttgatacctattcacctaacagcccggctcaatatgctcctgaaggtgatactcccaatgattatcccattacaggagctgcaaacgcacctaccggcatagatcccatagctgaaagccttgcttctgcttatggttctaaagccatataccaaatgcattggcttcttgacgtggataatttttataaattcggaaaccatggcgacggtacaagccgctgttcttatataaatacatatcagcgcgggcctgaagaatcggtttgggaaactgtaccgcacccatcctgggaagattttaaatggggtgaaggagaaaaaggcggttttttaaacctctttggcaattttggccagccggcaaaacaatggcgttacacttctgcctccgatgccgatgcaagacaggttcaagcctcctattgggcttatctttgggcaaaagaacaaggtgttgaaggagaactgtcagaatataccgataaagcagcaaaaatgggagattatttgagatatacattttttgataaatatttcagacccatcggtgttcaagacagttctgccgctggtacaggttatgacagctgccattatcttttatcatggtacatgtcctggggcggagatattggcggtgcatggagttggagaataggcagttcccactgccatcagggctatcaaaatcttgtagcagcctatgccctgtcacaggaacccgccttaaagccaaaggcacaaggatcgcaggaagactggcaaaaaagtttgaaacgccagattgaattgtatcagtacctccaaagtgcagaaggtgccattgcaggaggcgtcaccaacagttggcagggtagatattccaaatatcctgccggcaaaagcactttttacgatatggcctatgat

SEQ ID NO: 4 - DNA, 1062 bp, Clostridium sp.; identical to SEQ ID NO: 3

(19) United States
(12) Patent Application Publication
(10) Pub. No.: US 2012/0264183 A1
(43) Pub. Date: Oct. 18, 2012

(54) Cellulose and Xylan Fermentation by Novel Anaerobic Thermophilic Clostridia Isolated From Self-Heated Biocompost

(76) Inventors: Maria Sizova, Chestnut Hill, MA (US); Javier Izquierdo, Chapel Hill, NC (US); Lee R. Lynd, Meriden, NH (US)

(21) Appl. No.: 13/498,861

(22) PCT Filed: Sep. 28, 2010
(86) PCT No.: PCT/US10/50535

§ 371 Date: Jul. 5, 2012

Related U.S. Application Data
(60) Provisional application No. 61/246,440, filed on Sep. 28, 2009.

(51) Int. Cl. C12N 1/20; C12P 7/54; C12N 9/24; C12P 7/10; C12P 7/06; C12N 1/21; C12P 7/56; C12P 7/40

(52) U.S. Cl. 435/139; 435/252.7; 435/140; 435/136; 435/165; 435/161; 435/252.3; 435/200

(57) ABSTRACT

A new species of an anaerobic thermophilic cellulolytic and xylano lytic bacterium is disclosed. One particular strain of this new species has been deposited with the ATCC under Deposit No. PTA-10114. It is also provided a method for isolating, culturing and utilizing this novel bacterium for the conversion of biomass to bioconversion products, such as ethanol.

### RELATED APPLICATIONS

This application claims priority of U.S. Provisional Application No. 61/246,440 filed on Sep. 28, 2009, and U.S. Provisional Application No. 61/249,102 filed on Oct. 6, 2009, the contents of which are hereby incorporated into this application by reference.

### GOVERNMENT INTERESTS

The United States government may have certain rights in the present invention as research relevant to its development was funded by a grant DE-AC05-00OR22725 from the BioEnergy Science Center (BESC), a U.S. Department of Energy (DOE) Bioenergy Research Center supported by the Office of Biological and Environmental Research in the DOE Office of Science and by Mascoma Corp.

### SEQUENCE LISTING

This application is accompanied by a sequence listing both on paper and in a computer readable form that accurately reproduces the sequences described herein. These sequences have been deposited in GenBank under accession numbers FJ808599, FJ808600, GQ265352 and GQ265353.

### BACKGROUND

The present invention pertains to the field of biomass processing to produce ethanol and other products and more specifically, to the selection, isolation and use of novel anaerobic thermophilic cellulolytic and xylanolytic organisms. The invention relates to isolation of novel species of bacterium designated as Clostridium sp. 4-2a having ATCC deposit number PTA-10114. The Clostridium sp. strains 4-2a and 4-1 have been previously designated as a Clostridium polyfermentans strain 4-2a and strain 4-1, respectively. For purpose of consistency, these two strains have been re-designated as Clostridium sp. strain 4-2a and strain 4-1, respectively, and will be referred to under the new nomenclature throughout this disclosure.

Biomass represents an inexpensive and readily available cellulosic feedstock from which sugars may be produced. These sugars may be recovered or fermented to produce alcohols and/or other products. Among bioconversion products, interest in ethanol is high because it may be used as a renewable domestic fuel.

Cellulose and xylan present in biomass represent an inexpensive and readily available raw material from which sugars may be produced. These sugars may be used alone or fermented to produce alcohols and other products. Among bioconversion products, interest in ethanol is high because it may be used as a renewable domestic fuel. Bioconversion processes are becoming economically competitive with petroleum fuel technologies. Various reactor designs, pretreatment protocols, and separation technologies are known, for example, as shown in U.S. Pat. Nos. 5,258,293 and 5,837,506.

Several anaerobic thermophiles have been shown to utilize cellulose, including Clostridium thermocellum, C. straminisolvens, C. stercorarium, C. clariflavum and Caldicellulosiruptor saccharolyticus (Freier et al 1988; Kato et al. 2004; Madden 1983; Rainey et al. 1994; Shiratori et al. 2009).

The ultimate combination of biomass processing steps is referred to as consolidated bioprocessing (CBP). CBP involves four biologically-mediated events: (1) enzyme production, (2) substrate hydrolysis, (3) hexose fermentation and (4) pentose fermentation. These events may be performed in a single step by a microorganism that degrades and utilizes both cellulose and hemicellulose. Development of CBP organisms could potentially result in very large cost reductions as compared to the more conventional approach of producing saccharolytic enzymes in a dedicated process step. CBP processes that utilize more than one organism to accomplish the four biologically-mediated events are referred to as consolidated bioprocessing co-culture fermentations.

Among bacteria, Clostridia play an important role in anaerobic cellulose fermentation. Cellulolytic clostridia have been isolated from a wide variety of environments that are rich in decaying plant material such as soils, sediments, sewage sludge, composts, etc. (Leschine 2005).

C. thermocellum exhibits a high growth rate on crystalline cellulose (Lynd et al. 2002), but it does not utilize xylan. C. thermocellum does not grow on xylose or other pentoses, and grows poorly on glucose (Lynd et al. 2008). Extremely thermophilic cellulolytic Caldicellulosiruptor saccharolyticus can co-utilize glucose and xylose (van de Werken et al. 2008), while Anaerocellum thermophilum DSM 6725 has been found to degrade xylan and xylose by Yang et al (2009). However, the original report on this strain by Svetlichny et al (1990) showed that it did not utilize xylose. A. thermophilum has recently been shown to utilize cellulose and hemicellulose originating from lignocellulose with or without pretreatment (Yang et al., 2009). Cellulose conversion achieved by A. thermophilum cultures was <20%, although higher conversion was observed upon re-inoculation. Although several mesophilic Clostridium species have been reported to utilize both cellulose and xylan, including C. phytofermentas, C. cellulovorans (Warnick et al. 2002; Kosugi et al. 2001; Sleat et al. 1984), C. stercorarium is the only cellulolytic thermophilic Clostridium that has been reported to utilize both xylan and cellulose. One disadvantage of C. stercorarium is that its utilization of cellulose is modest as compared to C. thermocellum (Adelsberger et al. 2004; Zverlov and Schwartz 2008).

Microbial cellulose utilization is among the most promising strategies for biofuels production (Lynd et al. 2008a). After cellulose, xylan is the most predominant polymer in plants (Thompson 1993). Plant biomass represent an abundant and valuable renewable natural resource that may be put to wide range of uses, as a source of food, fiber chemicals, energy, etc. (Leschine 2005).

Isolation of novel microorganisms that are able to degrade major plant cell wall polymers such as cellulose, hemicelluloses and lignin, is essential for overcoming the recalcitrance of cellulosic biomass (Lynd et al. 2008b). Cellulolytic and xylanolytic Clostridium sp. strains 4-2a and 4-1 may be useful in processes for bioconversion of lignocelluloses to fuels, chemicals, protein, silage, biogas, etc.

### SUMMARY

The present instrumentalities advance the art and overcome the problems outlined above by providing methods for isolation and culture of cellulolytic microbes. By utilizing bacterial strains capable of metabolizing both cellulose and xylan containing material, these novel strains may serve as a source of thermostable xylanases and cellulases for industrial applications resulting in increased bioprocessing efficiency and economy.

More specifically, the present disclosure, provides a biologically pure culture of the Clostridium sp. strain 4-2a. Clostridium sp. strain 4-2a has been deposited, under the provisions of the Budapest Treaty, in the culture collection American Type Culture Collection (ATCC, Manassas, Va.) on Jun. 9, 2009 and bears the ATCC Deposit No. PTA-10114. It is also disclosed herein a second Clostridium sp. strain 4-1.

In an embodiment, an isolated biologically pure culture of an anaerobic thermophilic cellulolytic and xylanolytic bacterium bearing ATCC Deposit No. PTA-10114 is described.

In another embodiment, a biological material may be prepared which comprises an isolated biologically pure culture of an anaerobic thermophilic cellulolytic and xylanolytic bacterium bearing ATCC Deposit No. PTA-10114.

In another embodiment, the biological material of the present disclosure comprises an isolated biologically pure culture of an anaerobic thermophilic cellulolytic and xylanolytic bacterium which contains an endogenous gene having at least 70%, 80%, 90%, 95%, 99.9%, or most preferably, having 100% identity with SEQ ID No. 2.

In another embodiment, the biological material of the present disclosure comprises an isolated biologically pure culture of an anaerobic thermophilic cellulolytic and xylanolytic bacterium which contains a gene having at least 70%, 80%, 90%, 95%, 99%, or most preferably, having 100% identity with SEQ ID No. 4.

In another embodiment, the biological material of the present disclosure comprises an isolated biologically pure culture of an anaerobic thermophilic cellulolytic and xylanolytic bacterium which contains a functional exoglucanase having at least 70%, 80%, 90%, 95%, 99%, or most preferably, having 100% identity with the enzyme encoded by the polynucleotide sequence of SEQ ID No. 4.

In another embodiment, it is disclosed a functional exoglucanase having at least 70%, 80%, 90%, 95%, 99% or most preferably, having 100% sequence identity with the enzyme encoded by the polynucleotide sequence of SEQ ID No. 4.

In another embodiment, a polynucleotide having at least 70%, 80%, 90%, 95%, 99%, or most preferably, having 100% identity with SEQ ID No. 4 may be introduced into an organism and caused to be expressed in said organism in order to confer upon said organism the functionality similar to that of the exoglucanase of the new strain disclosed herein. By way of example, the polynucleotide may be introduced into the organism using transgenic or conjugation methods, among others. Such an organism may be called a transgenic organism, and the polynucleotide that is introduced into said organism may be called a transgene.

In a preferred embodiment, at least 50% of the artificially cultured biological material is the anaerobic thermophilic cellulolytic and xylanolytic bacterium bearing ATCC Deposit No. PTA-10114. Even more preferably, the cultured biological material contains at least 60%, 70%, 80%, 90% or 100% of the anaerobic thermophilic cellulolytic and xylanolytic bacterium bearing ATCC Deposit No. PTA-10114.

In an embodiment, a method for isolating a biologically pure culture of an anaerobic thermophilic cellulolytic and xylanolytic bacterium bearing ATCC Deposit No. PTA-10114 is described.

In another embodiment, a method for culturing an anaerobic thermophilic cellulolytic and xylanolytic bacterium bearing ATCC Deposit No. PTA-10114 is described.

It is also provided herein a method for conversion of a biomass to at least one bioconversion product. The method may include a step contacting the biomass with an isolated thermophilic cellulolytic and xylanolytic bacterium. In a preferred embodiment, the bacterium to be used contains an endogenous gene having at least 99.9% sequence identity with SEQ ID No. 2, or even more preferably, the bacterium is identical to the strain bearing ATCC Deposit No. PTA-10114. The biomass may be caused to be in contact with the disclosed bacterium in conjunction with at least one other bacterium. Alternatively, the contact between the biomass and the disclosed bacterium may be preceded and/or followed by another contacting step wherein the biomass is caused to be in contact with at least one other bacterium. The biomass may or may not have been pretreated before being caused to be in contact with the disclosed bacterium.

In another aspect, the biomass may be converted to the at least one bioconversion product by batch simultaneous saccharification and fermentation, by continuous culture, or by semi-continuous culture.

The biomass may contains a cellulosic material, a xylanosic material, a lignocellulosic material, or combination thereof. The bioconverion products may include but are not limited to lactic acid, formic acid, acetic acid, ethanol or mixture or salt thereof. In a preferred embodiment, the acetic acid/ethanol ratio in the final bioconverion products is at least 13.2.

### BRIEF DESCRIPTION OF DRAWINGS

FIG. 1 shows a diversity of colonies isolated and grown on Avicel-agar medium.

FIG. 2 is a phylogenetic tree of anaerobic thermophilic cellulolytic bacteria based on 16S rRNA gene sequence comparisons.

FIG. 3 is a phylogenetic tree of anaerobic thermophilic cellulolytic bacteria based on GHF48 gene sequence comparisons.

FIG. 4 is a graph depicting the dynamics of Avicel degradation and bacterial biomass growth in batch culture of strain 4-2a.

FIG. 5 is a graph depicting product formation of Avicel degradation in a batch culture of strain 4-2a.

FIG. 6 is a graph depicting the dynamics of xylan degradation and bacterial biomass growth in batch culture of strain 4-2a.

FIG. 7 is a graph depicting product formation of xylan degradation in a batch culture of strain 4-2a.

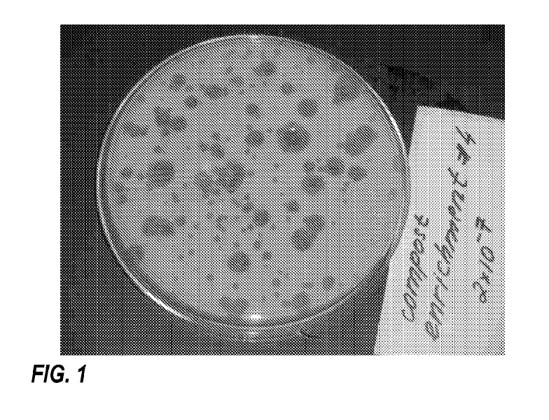

### DETAILED DESCRIPTION

There will now be shown and described a method for the isolation of novel cellulolytic and xylanolytic microbes.

As used herein, “cellulolytic” means capable of hydrolyzing cellulose.

As used herein, “xylanolytic” means capable of hydrolyzing xylan.

A biologically pure culture of an organism contains 100% of cells from said organism. As used herein, a “biologically pure culture” of bacteria is a genetically uniform culture of bacterial cells derived from a single colony. Such a culture contains 100% of cells that are progeny of the single colony. As used herein a culture may be a solid culture, or a liquid culture, such as but not limited to solid medium and liquid medium respectively. When referring to biological material or culture, the term “isolated” means the biological material or culture is prepared with some modification or the biological material or culture is purified from its naturally occurring sources.

As used herein, the term “biological material(s)” refers to bacteria, viruses, fungi, plants, animals or any other living organisms. For purpose of this disclosure, the biological material may contain a single biologically pure culture, or it may contain at least two genetically different cells from different strains that belong to the same or different species. For instance, the artificially cultured biological material of the present disclosure may be a mixture of a bacterial strain and a fungal strain. The biological material may be in a variety of forms, including but not limited to, liquid culture, solid culture, frozen culture, dry spores, live or dormant bacteria, etc. The term “artificially cultured” means that the biological material is grown for at least one cell cycle in a man-made environment, such as an incubator. The man-made environment may also be based on the natural environment of said biological material which has been modified to some degree to optimize the growth, reproduction and/or metabolism of the organism(s). It is to be recognized that the artificially cultured biological material may contain cells that are originally isolated from their natural environment.

As used herein, a biologically pure culture of Clostridium sp. 4-2a may be derived from Clostridium sp. strain 4-2a. Strains 4-2a may be purified via single colony isolation method.

As used herein, an organism is in “a native state” if it is has not been genetically engineered or otherwise manipulated by the hand of man in a manner that intentionally alters the genetic and/or phenotypic constitution of the organism. For example, wild-type organisms may be considered to be in a native state.

As used herein, thermophilic means capable of survival, growth and reproduction at temperatures greater than about 50° C.

Clostridium sp. strain 4-2a is an anaerobic thermophilic cellulolytic and xylanolytic gram positive bacterium.

Cellulase refers to a class of enzymes produced chiefly by fungi, bacteria, and protozoa that catalyze the cellulolysis (or hydrolysis) of cellulose.

As used herein, bioconversion products are the products that are generated by the breakdown of biomass. These products include, but are not limited to, ethanol, lactate, formate and acetate.

### Example 1

### Isolation of Clostridium sp. 4-2a

### Materials and Methods

Compost samples were collected at Middlebury College compost facilities in Middlebury Vt., USA. Samples were collected between 40 cm to 50 cm below the surface of the compost pile. The compost temperature varied between 52° C. and 72° C.

In contrast to previous studies, strictly anaerobic conditions were employed starting from primary sampling. Compost samples of between 8 g and 15 g were inoculated into bottles containing 100 ml of mineral medium, pH 7. One gram of Avicel (PH105; FMC Corp., Philadelphia, Pa.) was added to each bottle as a carbon source and flashed with nitrogen.

The primary mineral medium was formulated as follows: KH₂PO₄, 2.08 g/L; K₂HPO₄, 2.22 g/L; MgCl₂×6H₂O, 0.1 g/L; NH₄Cl, 0.4 g/L; CaCl₂×2H₂O, 0.05 g/L.

Upon arriving at the laboratory, the primary enrichments were brought to a temperature of 55° C. and incubated for 4 to 6 days. For consecutive transfers, defined minimal medium was prepared: Avicel, 3; KH₂PO₄, 1.04; K₂HPO₄, 1.11; NaHCO₃, 2.5; MgCl₂×6H₂O, 0.2; NH₄Cl, 0.4; CaCl₂×2H₂O, 0.05; FeCl₂×4H₂O, 0.05; L-cysteine HCl, 0.5; resazurin 0.0025. SL10-trace element, 1 ml/L (Atlas, 1996) and vitamin, 4 ml/L, solutions were added as concentrated solutions. The vitamin solution contained (g/l): pyridoxamine dihydrochloride, 0.2; PABA, 0.1; D biotin, 0.05; vitamin B12, 0.05; thiamine-HCl, 0.0125; folic acid, 0.5; Ca-pantothenate, 0.125; nicotinic acid, 0.125; pyridoxine-HCl, 0.025; thioctic acid, 0.125; riboflavin, 0.0125.

Phosphates and other minerals were prepared and autoclaved separately to avoid precipitation and unwanted chemical interactions during autoclaving. Vitamins were sterilized by filtration. Stock solutions (×100) of L-cysteine HCl, FeCl₂×4H₂O, MgCl₂×6H₂O; NH₄Cl; CaCl₂×2H₂O were flashed with N₂ immediately after dissolving and autoclaved. Serum bottles with sterile medium were placed into an anaerobic glove box, cooled down, mixed with reducing agent solution, closed with sterile rubber stoppers and caped with aluminum seals. To avoid contamination due to gas exchange during loading inside an airlock, all serum bottles were closed with sterile cotton balls and aluminum foil caps or rubber stoppers.

Descriptive statistics of primary data, including mean, confidence interval and standard deviation were done with MS Excel. 2-5 replicates were used for all analytical measurements (HPLC and TOCN) and relative error did not exceed 5%. The growth batch experiments were done at least twice with two replicate bottles. The time series data were used to calculate maximal specific growth rate and yield by using linear and non-linear regression with the Solver, MS Excel.

Phylogenetic trees were assembled using a bootstrap test with 1000 replicates to evaluate robustness.

To analyze Avicel, xylan, xylose and pretreated wood utilization products, anaerobic cellulolytic thermophilic strains were transferred into fresh defined medium with 3 g/l of related substrate. Batch cultures were incubated at 55° C. on shaker at 180 rpm for 2-7 days. Fermentation products were analyzed by HPLC at zero point and at the end of incubation.

### Isolation of Pure Cultures

Isolation of pure cultures of cellulose degrading bacteria was performed on agar-Avicel and agar-cellobiose media after 10 consecutive transfers of primary enrichments. The mineral composition was the same as described above. Vitamins were substituted with 2.0 g/l of yeast extract. Avicel was added at concentration 20 g/l, cellobiose at 10 and agar at 15 g/l. Cellulolytic consortium grown on defined Avicel medium was serially diluted into melted and cooled agar-Avicel medium (55° C. to 60° C.) and plated into Petri dishes inside an anaerobic glove box. After solidifying, the plates were incubated inside anaerobic jars at 55° C. Cellulose degrading bacteria formed zones of clearing in the Avicel-agar layer during incubation. Colonies were picked with a syringe needle and inoculated into defined Avicel and cellobiose liquid media. Isolates, primarily grown on cellobiose medium, were transferred onto Avicel-defined medium to assess their ability to degrade cellulose.

Two active cellulolytic strains 4-2a and 4-1 able to degrade cellulose, xylan and xylose were isolated from biocompost

### DNA Extraction, PCR Amplification, Sequencing and Alignment

Genomic DNA was extracted from microbial biomass with the GenElute Genomic DNA Kit (Sigma) according to manufactures instructions. PCR amplification of the 16s rRNA gene and sequencing was done as described before (Sizova et al. 2003). Amplification of GHF48 genes was performed with GH48F and GH48R degenerate primers (Izquierdo et al., 2010) Amplified PCR products were sequenced at Agencourt Bioscience Corporation, MA. Nucleotide sequences were aligned with sequences from GenBank using BioEdit v.7.0.5 (Hall 1999) and CLUSTALW (Thompson et al. 1994).

### Phylogenetic Analysis of Bacterial Isolates

Phylogenetic trees were reconstructed using the ME-algorithm (Rzhetsky and Nei 1992) via the MEGA4 program package (Tamura et al. 2007). Screening for similarity was carried out with BLAST.

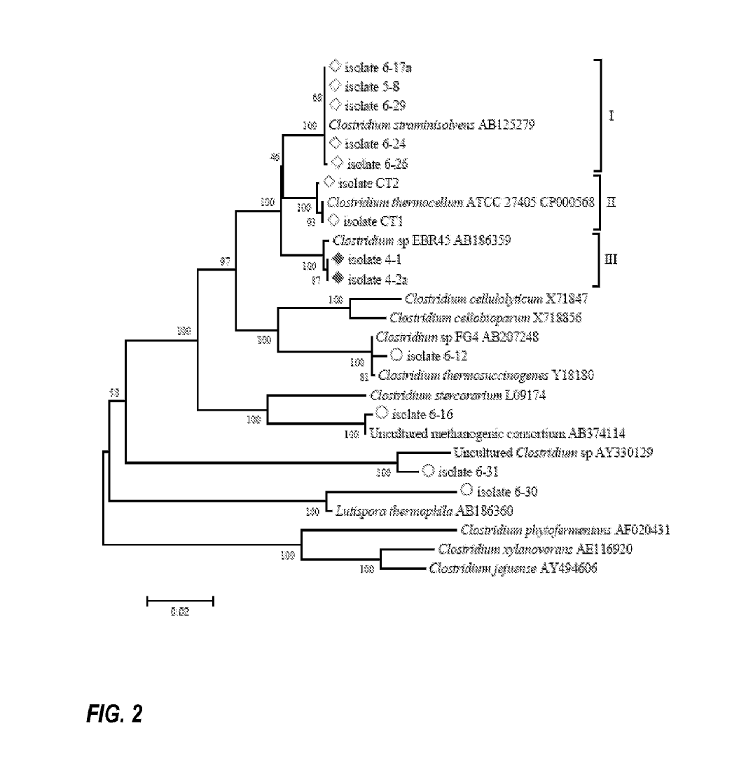

FIG. 2 shows a phylogenetic tree of anaerobic thermophilic cellulolytic bacteria based on 16S rRNA gene sequence comparisons. Phylogenetic analysis revealed that isolated strains 4-1 and 4-2a are most closely related to novel Clostridium clariflavum that actively fermented paper waste in thermophilic methanogenic reactor (Shiratori et al. 2006; Shiratori et al. 2009). The sequences of 16S rRNA from 4-1 (SEQ ID No. 1) and 4-2a (SEQ ID No. 2) have been deposited with GenBank and have been assigned accession numbers FJ808599 and FJ808600, respectively.

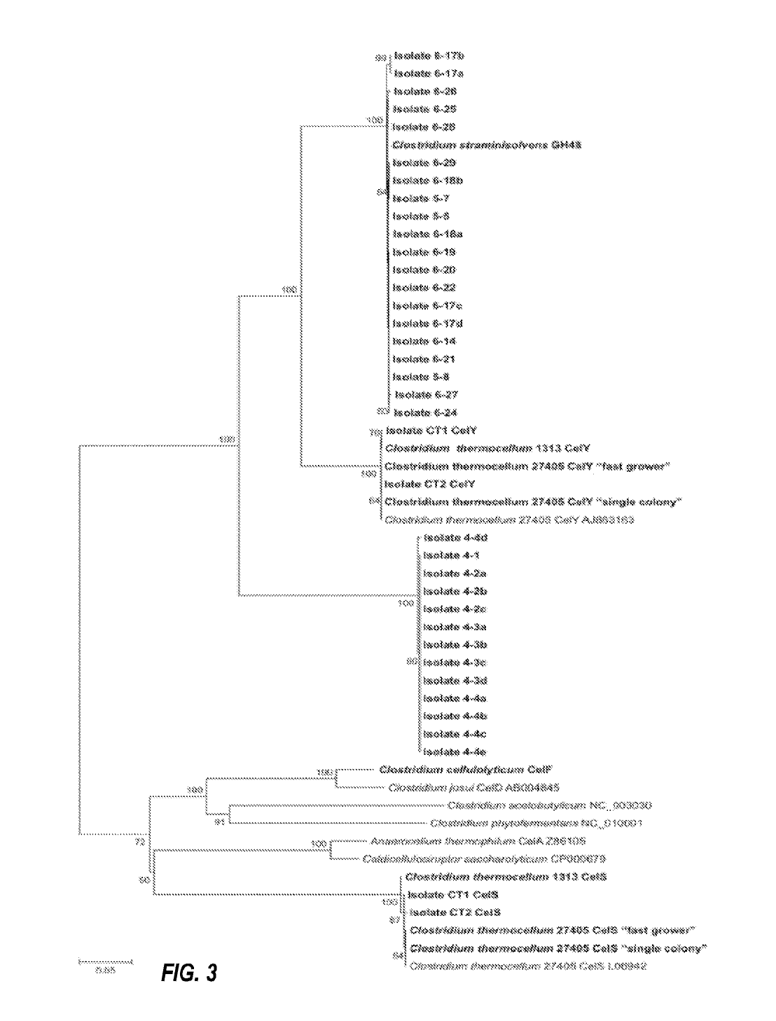

FIG. 3 is a phylogenetic tree of anaerobic thermophilic cellulolytic bacteria based on GHF48 gene sequence comparisons.

Glycoside hydrolases (GHs) (EC 3.2.1.) are a widespread group of enzymes which hydrolyze the glycosidic bond between two or more carbohydrates or between a carbohydrate and a non-carbohydrate moiety. The IUB-MB enzyme nomenclature of glycoside hydrolases is based on their substrate specificity and occasionally on their molecular mechanism; such a classification does not reflect the structural features of these enzymes.

In most cases, the hydrolysis of the glycosidic bond is performed by two catalytic residues of the enzyme: a general acid (proton donor) and a nucleophile/base. Depending on the spatial position of these catalytic residues, hydrolysis occurs via overall retention or overall inversion of the anomeric configuration.

Phylogenetic analysis was also carried out with respect to exocellulases of glycosyl hydrolase family 48 (GHF48), a major enzyme of interest within cellulolytic microorganisms. Clostridium sp. strains 4-1 and 4-2a, formed a distinct cluster of identical nucleotide sequences with no known sequences closely related to them. The closest matches were C. thermocellum CelY (74.1% similarity in nucleotide sequence, 87% translated amino acid sequence similarity) and C. straminisolvens (73.4% similarity in nucleotide, 87% translated amino acid sequence similarity). The translated amino acid sequence of GHF48 enzymes from 4-1 or 4-2a may be obtained by translating the GHF48 gene sequences from 4-1 (SEQ ID No 3) or from 4-2a (SEQ ID No 4) using standard genetic codes.

GHF48 genes in Clostridium sp. strains 4-2a and 4-1 displayed a very similar grouping as observed in 16S rRNA gene analyses, suggesting a very strict conservation of this particular family of glycosyl hydrolases within cellulolytic Clostridia. GHF48 sequences isolated from strains 4-1 (SEQ ID No. 3, GenBank Accession #GQ265352) and 4-2a (SEQ ID No. 4, GenBank Accession #GQ265353) have been deposited with GenBank. These GHF48 genes encode proteins which represent novel exoglucanases that may be useful in the biofuel industry.

### Fermentation Physiology

To analyze Avicel, xylan, xylose and pretreated wood utilization products, anaerobic cellulolytic thermophilic strains were transferred into fresh defined medium with 3 g/l of related substrate. Batch cultures were incubated at 55° C. on a shaker at 180 rpm. Fermentation products were analyzed by HPLC with an Aminex HPX-87H column (Bio-Rad Laboratories) at zero point and at the end of incubation. Major products of Avicel, xylan, xylose and pretreated wood fermentation are shown in Table 1. Major fermentation products of Avicel were acetate and formate, with lactate accumulating at the late stage of fermentation (FIG. 5)

It was observed that xylan was degraded during the first day of incubation while accumulation of pretreated wood and xylose fermentation products took between 5-7 days. In contrast to the fermentation products formed from pretreated wood, i.e. acetate and lactate, the major fermentation products of xylan were acetate and formate. Ethanol concentrations varied from 0.6 to 1.1 mM with the acetate to ethanol ratio being 10.9-19.3. Both the 4-1 and 4-2a isolates were able to use xylose as a single source of carbon. Microbial growth on xylose was much slower than on Avicel, xylan and pretreated wood. Only ˜50% of xylose was fermented during 10 days of incubation. The major fermentation product of xylose was acetate and lactate, no ethanol was detected

TABLE 1
Fermentation products formed by isolates 4-1 and 4-2a from
Avicel, xylan, pretreated wood and xylose (3 g/l).

| | | | | | | Acetate/ |
|---|---|---|---|---|---|---|
| | Isolate | Lactate | Formate | Acetate | Ethanol | Ethanol |
| Substrate | mM | | | | | ratio |
| Avicel | 4-1 | 0.2 | 2.7 | 7.8 | 0.3 | 22.2 |
| | 4-2a | 1.0 | 3.5 | 9.2 | 0.9 | 10.3 |
| Xylan | 4-1 | 0.3 | 3.6 | 12.8 | 0.7 | 18.6 |
| | 4-2a | 0.5 | 3.0 | 12.2 | 0.6 | 19.3 |
| Pretreated | 4-1 | 2.1 | 0.7 | 11.6 | 1.1 | 10.9 |
| wood | 4-2a | 1.0 | 0.1 | 10.4 | 0.8 | 13.2 |
| Xylose | 4-1 | 0.1 | | 2.1 | | |
| | 4-2a | 0.5 | | 2.7 | | |

Two isolated strains, 4-1 and 4-2a, were able to degrade cellulose, xylan and xylose. These two cellulolytic and xylanolytic strains were related to Clostridium clariflavum.

### Dynamics of Cellulose and Xylan Utilization

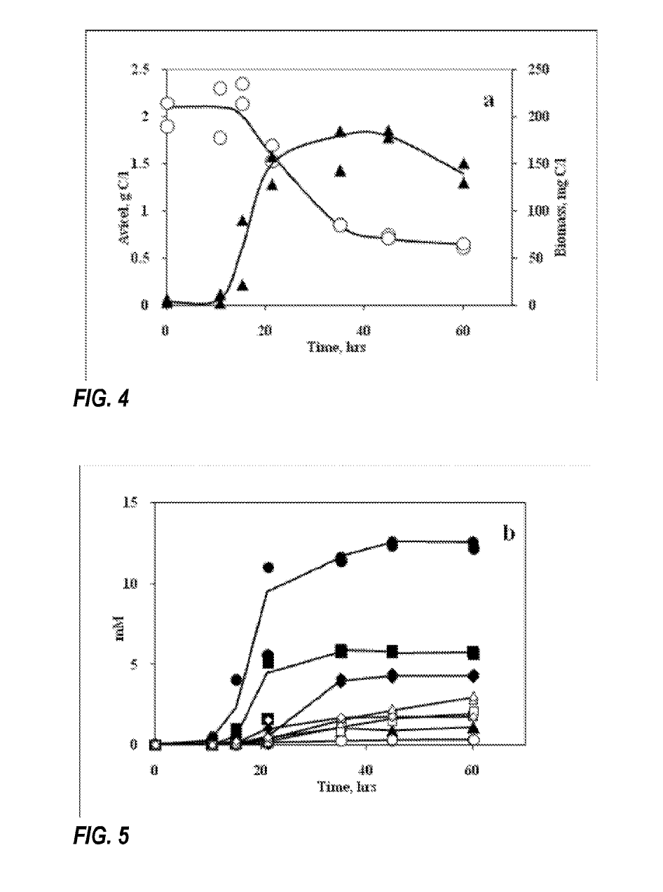

One percent of freshly grown culture was used as inoculums. Degradation of Avicel began after a lag period of about 11-15 hr. FIG. 4 shows that about 60% of Avicel was utilized during 10-15 hrs of exponential growth of strain 4-2a (symbols: o, concentration of Avicel; ▴, cells biomass). Bacterial biomass accumulated exponentially during first 21 hrs. Approximate biomass yield was about 0.13 mg C-biomass/mg C-Avicel. The degradation process abruptly ceased as the pH of the culture medium dropped from pH 8 to pH 6. pH was measured using an Ultra Basic Bench top pH meter UB-10 (Denver Instrument).

The major fermentation products were acetate, formate, lactate and ethanol. As shown in FIG. 5, acetate, formate and ethanol were formed exponentially in parallel with bacterial growth (symbols: , acetate; ▪, formate; ▴, ethanol; ♦, lactate; o, xylose; □, cellobiose; Δ, glucose; ⋄, glycerol). It was observed that, as pH declined, lactate, cellobiose, glucose, glycerol and xylose accumulated in the cultural medium. At the end of incubation the acetate/ethanol ratio was about 12:1.

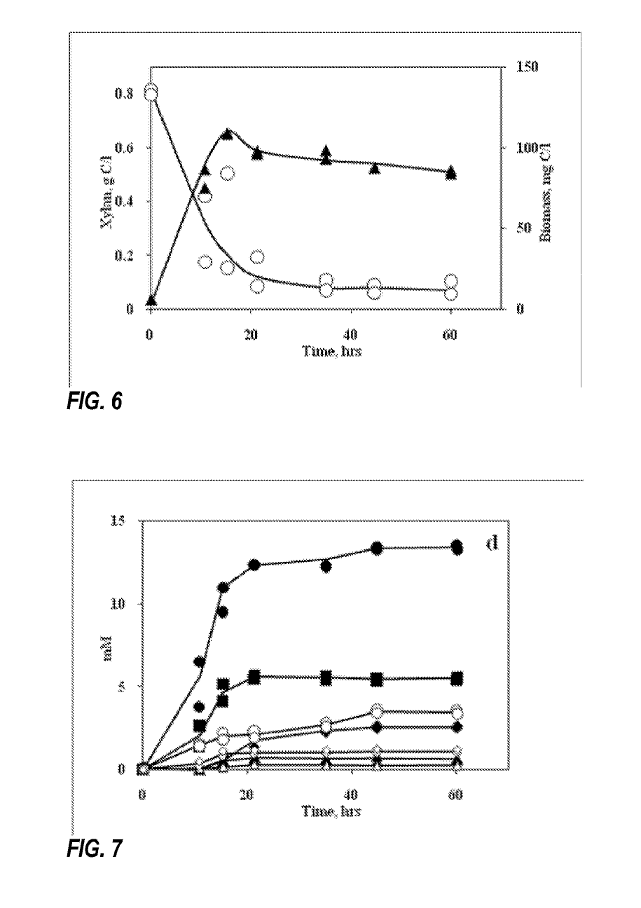

FIG. 6 is a graph illustrating the dynamics of xylan degradation in batch cultures of strain 4-2a (symbols: o, concentration of xylan; ▴, cells biomass). Degradation of xylan began immediately after inoculation. During the first 21 hrs of incubation about 75% of xylan was degraded, while bacterial biomass and accumulation of fermentation products and intermediates increased (FIG. 7; symbols: , acetate; ▪, formate; ▴, ethanol; ♦, lactate; o, xylose; Δ, glucose; ⋄, glycerol). During incubation, pH declined (data not shown).

Approximate biomass yield on xylan was 0.14 mg C-biomass/mg C-xylan, comparable to biomass yield on Avicel. The degradation process stopped as pH decreased from about pH 8 to about pH 6.3. The major fermentation products acetate, formate, lactate as well as the xylose, glucose and glycerol intermediates accumulated over time. The concentration of intermediate xylose reached 3.5 mM, while ethanol concentration reached only 0.6 mM during 60 hrs of incubation. The acetate/ethanol ratio was about 22:1.

Clostridium sp. strains 4-2a and 4-1 represent a new anaerobic, thermophilic and cellulolytic organism within the Clostridium genus, besides C. stercorarium (Adelsberger et al. 2004) that is capable of degrading cellulose, xylan and xylose.

Description of Clostridium sp. Strains 4-2a and 4-1.

Clostridium sp. strains 4-2a and 4-1 cells are straight and slightly curved rods 3-12×0.1-0.3 μm when grown on Avicel and straight rods 3-5×0.2-0.3 μm when grown on xylan. Clostridium sp. strain 4-2a and 4-1 forms terminal spores. Surface colonies (in agar-cellobiose medium) are extremely slimy and light cream colored. Colonies grown in agar-Avicel medium produce 5-10 mm zones of clearing during 7 days of incubation. Clostridium sp. strain 4-2a and 4-1 is an obligate anaerobe. Bacterial cultures of Clostridium sp. strain 4-2a and 4-1 robustly grow on Avicel or xylan as a single carbon source. Biomass yield is 0.13 mg C-biomass/mg C-Avicel with N/C ratio 0.27. Major fermentation products were acetate, formate, lactate and ethanol. Clostridium sp. strain 4-2a and 4-1 grows on cellobiose and partially ferments xylose. Growth occurs at temperature 55-60° C. and pH 6.0-8.0.

Adaptation of traditional plating techniques allowed for the isolation of new anaerobic thermophilic bacteria that utilize cellulose.

Microbial culture purification and identification requires the isolation of a single colony. Consistent results were observed when consortia grown in cellulose liquid medium till the middle of log phase were plated within agar layer. It was important to make all manipulations inside of anaerobic glove box and prepare serial dilutions in nutrient medium but not sterile water.

The major methodological principle was to mimic natural conditions of anaerobic cellulose degradation in situ. Conditions that were crucial in this process were: a) strictly anaerobic conditions starting from primary sampling; b) cellulose (Avicel or filter paper) as the only source of carbon and energy (no yeast extract or vitamins were added); c) enrichment incubation temperature was the same as in situ; d) nitrates, sulfates, sulfides were excluded to avoid the development of competitive microorganisms.

Thus, anaerobic sampling procedures in combination with adapted plating techniques allows for the isolation of novel cellulolytic microorganisms even from very well studied environments like biocompost piles. Biocompost remains one of the most promising natural environments for isolation of active plant biomass degraders.

Microbial cellulose utilization is among the most promising strategies for biofuels production (Lynd et al. 2008). Plant biomass represents an abundant and valuable renewable natural resource that may be put to wide range of uses, as a source of food, fiber chemicals, energy, etc (Leschine 2005). Novel cellulolytic and xylanolytic strains described in this study can serve as potential source of previously unknown thermo stable xylanases and cellulases for plant biomass conversion and other industrial applications. After cellulose, xylan is the most predominant polymer in plants (Thompson 1993). Microorganisms and enzymes actively fermented plant polymers are extremely useful for a broad range of environmentally friendly industrial processes. Microbial xylanases assume special importance in the paper and pulp industry as they help to minimize the use of toxic chemicals (Kulkarni et al. 1999). Xylanases are also used as nutritional additives to silage and grain feed, for the extraction of coffee and plant oils and in combination with pectinases and cellulases for clarification of fruit juices (Beg et al. 2001).

Therefore, cellulolytic and xylanolytic strains described above are useful for further characterizing cellulase and xylanase diversity as well as in processes for bioconversion of lignocelluloses to fuels, chemicals, protein, silage, biogas, etc.

### Example 2

### Preparation of Cultivation Medium

Two different solutions of chemicals were prepared separately in order to avoid precipitation and chemical interactions during autoclaving. Vitamins were sterilized by filtration.

Preparation of a 1000× solution of trace elements SL-10 is described in Table 2.

TABLE 2
Trace element solution SL-10 (1000X)

| Component | Amount | |
|---|---|---|
| HCl (25%) | 10 | ml |
| FeCl₂x4H₂O | 1.5 | g/l |
| CoCl₂x6H₂O | 0.19 | g/l |
| MnCl₂x4H₂O | 0.1 | g/l |
| ZnCl₂ | 0.07 | g/l |
| Na₂MoO₄x2H₂O | 0.036 | g/l |
| NiCl₂x6H₂O | 0.024 | g/l |
| H₃BO₃ | 0.006 | g/l |
| CuCl₂x2H₂O | 0.002 | g/l |

Preparation of a 250× solution vitamins is described in Table 3.

TABLE 3
Vitamin solution (250X)

| Component | Amount g/l |
|---|---|
| Pyridoxamine Dihydrochloride | 0.2 |
| Para-aminobenzoic acid (PABA) | 0.1 |
| D Biotin | 0.05 |
| Vitamin B 12 | 0.05 |
| Thiamine HCl | 0.0125 |
| Folic Acid | 0.05 |
| Pantotenic acid-Ca⁺⁺ salt | 0.125 |
| Nicotinic acid | 0.125 |
| Pyridoxine-HCl | 0.025 |
| Thioctic acid | 0.125 |
| Riboflavin | 0.0125 |

Preparation of solution A is described in Table 4.

TABLE 4
Solution A

| Components | Final Amount | |
|---|---|---|
| Avicel | 3.0 | g/l |
| KH₂PO₄ | 1.04 | g/l |
| K₂HPO₄ | 1.11 | g/l |
| Trace Elements SL-10 | 1 | ml |
| NaHCO₃ | 2.0 | g/l |
| Resazurin 0.025% | 0.01 | g/l |

Preparation of a 100× stock solution B is described in Table 5.

TABLE 5
Solution B (100X)

| Components | Final Amount | Stock solution, g/l |
|---|---|---|
| NH₄Cl | 0.4 g/l | 4.0 |
| MgCl₂x6H₂O | 0.1 g/l | 1.0 |
| CaCl₂xH₂O | 0.05 g/l | 0.5 |
| L-cysteine HCl: | 0.5 g/l | 5.0 |
| C₃HNO₂SxHClxH₂O | | |
| FeCl₂x4H₂O | 0.05 g/l | 0.5 |

Medium was prepared by preparing solution A and distributing solution A into serum bottles. Serum bottles were closed with rubber stoppers and sealed with aluminum caps. Bottles were then flashed with nitrogen. L-cysteine HCL and FeCl₂×4H₂O were dissolved and mixed with the additional components of solution B in a serum bottle. The bottle was closed with a rubber stopper and sealed with an aluminum cap. The serum bottle was immediately flashed with nitrogen. All serum bottles were then autoclaved for 20-25 min Sterile anaerobic stock solution B and vitamin solution was then aseptically transferred to serum bottles containing solution A using a sterile needle and syringe. After about 10-20 minutes the combined solutions became colorless.

The disclosed microbes may be utilized in a consolidated bioprocessing (CBP) process with no added enzymes. Methods of utilizing cellulolytic microbes for the conversion of cellulosic material into ethanol are known. Cellulosic materials that may be converted by the presently described microbes include any feedstock that contains cellulose, such as wood, corn, corn stover, sawdust, bark, leaves, agricultural and forestry residues, grasses such as switchgrass or miscanthus or mixed prairie grasses, ruminant digestion products, municipal wastes, paper mill effluent, newspaper, cardboard or combinations thereof.

### Example 3

### Simultaneous Saccharification and Fermentation

As discussed above, the thermophilic organism Clostridium sp. strain 4-2a and 4-1 has the potential to contribute significant savings in lignocellulosic biomass to ethanol conversion due to their ability to utilize cellulose, xylose and xylan.

Clostridium sp. strains 4-2a and 4-1 are used to produce ethanol and other products in the bioconversion processes of consolidated bioprocessing (CBP)

It will be appreciated that Clostridium sp. strain 4-2a and 4-1 can ferment both pentose and hexose sugars.

### Batch SSF and Relevant Enzyme Controls.

Five ml of a Clostridium sp. 4-2a (ATCC Deposit No. PTA-10114) stock culture is inoculated into 100 ml medium containing a 3 grams of a carbon source and under a N2 atmosphere. The carbon source may be Avicel, xylan, pretreated wood, or xylose or a combination thereof. Cultures are incubated at 55° C. in a temperature controlled water bath with rotary shaking at 180 rpm. pH is adjusted to 8.

### Continuous Culture.

The reaction vessel was a modified 1 L fermentor (Applikon, Dependable Instruments, Foster City, Calif., modified by NDS) with an overflow sidearm (i.d. 0.38″) and 0.5 L working volume is used for both microbial fermentation by Clostridium sp. 4-2a (ATCC Deposit No. PTA-10114) and for SSF carried out in continuous mode. pH was controlled by a Delta V process control system (New England Controls Inc., Mansfield, Mass.) with addition of 4M NaOH, the fermentor was stirred at between 180 rpm and 250 rpm, and temperature was controlled at 55° C. by circulating hot water through the fermentor jacket. Medium containing 3 g/L Avicel, xylan, pretreated wood, or xylose or a combination thereof is fed by a peristaltic pump to achieve the desired residence times. SSF experiments are initiated by inoculating 50 ml of a late-exponential phase culture of Clostridium sp. 4-2a (ATCC Deposit No. PTA-10114) into medium containing 3 g/L Avicel, xylan, pretreated wood, or xylose or a combination thereof. Samples used to calculate steady-state values for continuous fermentations are taken at intervals of at least one residence.

### Strain Deposit

Clostridium sp. strain 4-2a has been deposited with the American Type Culture Collection, Manassas, Va. 20110-2209. The deposit was made on Jun. 9, 2009 and received Patent Deposit Designation Number PTA-10114. This deposit was made in compliance with the Budapest Treaty requirements that the duration of the deposit should be for thirty (30) years from the date of deposit or for five (5) years after the last request for the deposit at the depository or for the enforceable life of a U.S. patent that matures from this application, whichever is longer. Clostridium sp. 4-2a will be replenished should it become non-viable at the depository.

The description of the specific embodiments reveals general concepts that others can modify and/or adapt for various applications or uses that do not depart from the general concepts. Therefore, such adaptations and modifications should and are intended to be comprehended within the meaning and range of equivalents of the disclosed embodiments. It is to be understood that the phraseology or terminology employed herein is for the purpose of description and not limitation. All references mentioned in this application are incorporated.

What is claimed is:

1. A biological material comprising an isolated anaerobic thermophilic cellulolytic and xylanolytic bacterium, said bacterium comprising an endogenous gene having at least 99.9% sequence identity with SEQ ID No. 2.

2. The biological material of claim 1 wherein said endogenous gene has 100% sequence identity with SEQ ID No. 2.

3. The biological material of claim 1 wherein said bacterium is identical to the bacterium bearing ATCC Deposit No. PTA-10114.

4. The biological material of claim 1 wherein said bacterium further comprises a functional exoglucanase having at least 80% sequence identity with the enzyme encoded by the polynucleotide sequence of SEQ ID No. 4.

5. The biological material of claim 4 wherein said bacterium further comprises a functional exoglucanase having at least 95% sequence identity with the enzyme encoded by the polynucleotide sequence of SEQ ID No. 4.

6. The biological material of claim 4 wherein said bacterium further comprises a functional exoglucanase having at least 99% sequence identity with the enzyme encoded by the polynucleotide sequence of SEQ ID No. 4

7. A method for conversion of a biomass, said method comprising contacting said biomass with an isolated thermophilic cellulolytic and xylanolytic bacterium, said bacterium comprising an endogenous gene having at least 99.9% sequence identity with SEQ ID No. 2.

8. The method of claim 7, wherein said bacterium comprises an endogenous gene having 100% sequence identity with SEQ ID No. 2.

9. The method of claim 7, wherein said bacterium is identical to the bacterium bearing ATCC Deposit No. PTA-10114.

10. The method of claim 7 wherein the biomass is converted to at least one bioconversion product by batch simultaneous saccharification and fermentation.

11. The method of claim 7 wherein the biomass is converted to at least one bioconversion product by continuous culture.

12. The method of claim 7 wherein the biomass is converted to at least one bioconversion product by semi-continuous culture.

13. The method of claim 7 wherein the biomass comprises a cellulosic material.

14. The method of claim 7 wherein the biomass comprises a xylanosic material.

15. The method of claim 7 wherein the at least one bioconverion product is selected from the group consisting of lactic acid, formic acid, acetic acid, ethanol and combination or salt thereof.

16. The method of claim 15 wherein an acetic acid/ethanol ratio is at least 13.2.

17. A transgenic organism comprising a transgene, said transgene comprising a polynucleotide having at least 80% sequence identity with SEQ ID No. 4.

18. The transgenic organism of claim 17 wherein said polynucleotide encodes a functional exoglucanase having at least 95% sequence identity with the enzyme encoded by the polynucleotide sequence of SEQ ID No. 4.

19. The transgenic organism of claim 17, wherein said exoglucanase has 100% sequence identity with the enzyme encoded by the polynucleotide sequence of SEQ ID No. 4.

20. An isolated biologically pure culture of an anaerobic thermophilic cellulolytic and xylanolytic bacterium bearing ATCC Deposit No. PTA-10114.

21. An isolated cellulolytic and xylanolytic bacterium bearing ATCC Deposit No. PTA-10114.

22. A protein molecule having at least 95% sequence identity with the enzyme encoded by the polynucleotide sequence of SEQ ID No. 4.
